(19) United States
(12) Patent Application Publication
(10) Pub. No.: US 2011/0208052 A1
(43) Pub. Date: Aug. 25, 2011

(54) BREAST ULTRASOUND ANNOTATION USER INTERFACE

(75) Inventor: Robert R. Entrekin, Kirkland, WA (US)

(73) Assignee: KONINKLIJKE PHILIPS ELECTRONICS N.V., Eindhoven (NL)

(21) Appl. No.: 13/125,814

(22) PCT Filed: Oct. 16, 2009
(86) PCT No.: PCT/IB2009/054558

§ 371 Date: Apr. 25, 2011

Related U.S. Application Data
(60) Provisional application No. 61/111,807, filed on Nov. 6, 2008.

(51) Int. Cl. A61B 8/00; G06F 17/00

(52) U.S. Cl. 600/437; 715/232; 715/231

(57) ABSTRACT

The present invention refers to a graphical user interface (501) and a corresponding method for rapid and consistent input, modification and display of annotation text to be linked with and displayed in at least one image visualized on a monitor screen or display (102) of a medical or other kind of imaging system (100) without needing to type this text information (e.g. by activating or deactivating softkeys on a touch screen (502) or by rotary knob selection). Additionally, said user interface (501) allows to automatically link graphical annotation information, such as e.g. body markers and graphical transducer orientation information, to the annotated text.

### FIELD OF THE INVENTION

The present invention refers to a graphical user interface and a corresponding method for rapid and consistent input, modification and display of annotation text to be linked with and displayed in at least one image visualized on a monitor screen or display of a medical or other kind of imaging system without needing to type this text information (e.g. by activating or deactivating softkeys on a touch screen or by rotary knob selection). Additionally, said user interface allows to automatically link graphical annotation information, such as e.g. body markers and graphical transducer orientation information, to the annotated text. The proposed method is faster and easier than conventional methods of keyboard text entry, eliminates the problem of discordant annotation text and body marker orientation due to user oversight or error and can advantageously be applied in the field of medical imaging applications (e.g. such as, inter alia, in the field of breast ultrasound imaging). Although the specific embodiment described in this application disclosure merely refers to 2D breast imaging, the proposed method can also beneficially be applied to 3D breast imaging and, aside from ultrasound imaging, to any other kind of clinical imaging applications and medical user interfaces.

### BACKGROUND OF THE INVENTION

Breast ultrasound imaging, also known as breast sonography or ultrasonography, is frequently used to evaluate tissue abnormalities in the interior of the female breast that have been found with screening or diagnostic mammography or during a physician performed clinical breast exam. Ultrasound imaging thereby allows significant freedom in obtaining images of the breast from almost any orientation. Furthermore, ultrasound may help to detect suspicious breast masses and is the best way to determine whether a cyst is present without placing a needle into a tissue region of interest to aspirate fluid. Aside from the fact that ultrasound is excellent at imaging cysts, which can be found as round, fluid-filled, pockets inside a female breast, sonography can often help to quickly determine whether a suspicious region within a woman's breast tissue is in fact a cyst (always non-cancerous), a lymph node or an increased density of solid tissue (dense mass) which may require a biopsy to determine if it is cancerous (benign or malignant). Ultrasound imaging is also useful in helping physicians guide a biopsy (tissue sampling) to determine whether a breast abnormality is cancerous. Physicians also use ultrasound imaging during core and fine needle aspiration biopsies (FNA) to determine where to place the needle.

US 2006/0174065 A1 describes a system and method for annotating data displayed on a display device. The system includes a processing unit for processing data and providing the processed data to the display device for displaying a portion thereof, and further generating a marker (cursor) for display by the display device and accessing a data set including a plurality of labels. The system further includes a user input device for transmitting a series of user request signals to the processing unit upon manipulation of the user input device with a user's hand, and a switch in proximity to the user input device for transmitting mode selection signals to the processing unit for selecting one of a cursor movement mode and an annotation mode. The switch is located sufficiently proximate the user input device for being selectively switched by the user's hand during manipulation of the user input device. The user input device can thereby be used in combination with said switch to either position said cursor or select an annotation text from a predefined list that is supposed to be positioned close to the cursor. When the cursor movement mode is selected, the series of user request signals control movement of the cursor on the display. When the annotation mode is selected, the series of user request signals control selection of a label of the plurality of labels for display at approximately the current cursor location for annotating the displayed data.

WO 2006/038182 A1 describes an ultrasonic diagnostic imaging system which is used for acquiring an ultrasound image of a region of interest in the interior of a patient's body that is assumed to contain diseased tissue, such as e.g. a suspected lesion, a tumor, etc. A body marker template of said region of interest is displayed on a touchscreen display of the imaging system such that an operator is able to record the location of the suspect anatomy by touching a corresponding point on the body marker template displayed on the touchscreen display and thus to indicate the location of a suspicious anatomy. The position and orientation of the marker on the template can then be finely adjusted by one or more controls on the imaging system control panel. The body marker template can also record a graphic indicating the orientation of the ultrasound probe relative to the body when the suspect anatomy was imaged. A report generator produces a report containing both the ultrasound image of the suspect anatomy and the body marker template with the indicated location of the suspect anatomy.

### SUMMARY OF THE INVENTION

Use of keyboard text entry is laborious and time-consuming, slowing down workflow and patient's throughput. Furthermore, having two separate user interfaces for annotation text and graphical annotation information (such as e.g. for inputting graphical symbols which may e.g. denote distinct body markers and transducer orientation labels) creates extra processing steps for the user and may thus create opportunities for errors and inconsistencies between the annotation text and the body marker indication.

In view of the above-described facts, it is an object of the present invention to provide a unified user interface for both text and graphical annotation, which allows a faster, easier, and more accurate workflow than conventional annotation input devices and methods.

Therefore, a first exemplary embodiment of the present invention is directed to a graphical user interface for enabling input, modification, display, outfading and cancellation of annotation text and graphical annotation information which, when being selected by a user, are linked with the image data of at least one image on an imaging system's monitor screen or display and displayed at user-definable positions within a region of interest of said at least one image and which, when being deselected by the user, are cancelled or faded out. According to this embodiment, it is thereby provided that the annotation text and said graphical annotation information are linked and depend on each other. Furthermore, it is provided that the graphical user interface is configured such that, if a specific annotation text is entered, selected or changed by the user, a corresponding graphical annotation information is automatically selected or accordingly modified so as to be consistent with the selected annotation text and displayed together with this selected annotation text on the monitor screen or display and if a specific graphical annotation information is selected or changed by the user, a corresponding annotation text is automatically selected or accordingly modified so as to be consistent with the user-selected graphical annotation information and displayed together with this user-selected graphical annotation information on the monitor screen or display.

The graphical user interface may thereby be configured such that the annotation text and graphical annotation information can e.g. be selected or deselected by activating or deactivating softkeys on a touch screen or by using at least one rotary knob for selecting a desired annotation text or graphical annotation information from at least one pull-down menu or scrollable list.

Preferably, it may be provided that the graphical user interface as proposed above is applied in the scope of a breast ultrasound imaging application where said annotation text is given by at least one text label for indicating the orientation of an ultrasound transducer or said ultrasound transducer's image plane relative to a patient's body during an ultrasound examination of said patient.

The graphical annotation information may e.g. be given by body marker symbols, pictograms or polar plots (“clock diagrams”) to be displayed on the monitor screen or display for visually indicating the location of at least one pathological tissue anomaly or lesion or visually indicating the angular orientation of said ultrasound transducer during an ultrasound examination relative to the patient while being slidably moved over the surface of said patient's body.

According to a specific aspect of said first exemplary embodiment, said graphical user interface may e.g. be configured such that annotation text and graphical annotation information indicating whether an ultrasound image of the patient's left or right breast, indicating the number of said pathological tissue anomalies or lesions or indicating the radii or angles of their polar coordinates, with the nipple position of said breast being defined as the origin of a polar coordinate system to which said polar coordinates refer, can be selected by rotary knob selection from a number of pull-down menus or scrollable lists.

A second exemplary embodiment of the present invention refers to an imaging system for acquiring, processing and displaying image data to be visualized on a monitor screen or display, wherein said imaging system comprises a graphical user interface as described with reference to said first exemplary embodiment.

A third exemplary embodiment of the present invention is dedicated to a method for enabling input, modification, display, outfading and cancellation of annotation text and graphical annotation information which, when being selected by a user, are linked with the image data of at least one image on an imaging system's monitor screen or display and displayed at user-definable positions within a region of interest of said at least one image and which, when being deselected by the user, are cancelled or faded out. According to this embodiment, it is again provided that the annotation text and said graphical annotation information are linked and depend on each other. Furthermore, said method provides that, if a specific annotation text is entered, selected or changed by the user, a corresponding graphical annotation information is automatically selected or accordingly modified so as to be consistent with the selected annotation text and displayed together with this selected annotation text on the monitor screen or display and if a specific graphical annotation information is selected or changed by the user, a corresponding annotation text is automatically selected or accordingly modified so as to be consistent with the user-selected graphical annotation information and displayed together with this user-selected graphical annotation information on the monitor screen or display.

As already described with reference to said first exemplary embodiment, it can be provided that according to this method said annotation text and graphical annotation information can be selected or deselected by activating or deactivating softkeys on a touch screen or by using at least one rotary knob for selecting a desired annotation text or graphical annotation information from at least one pull-down menu or scrollable list.

The proposed method as described above may preferably be applied in the scope of a breast ultrasound imaging application where said annotation text is given by at least one text label for indicating the orientation of an ultrasound transducer or said ultrasound transducer's image plane relative to a patient's body during an ultrasound examination of said patient.

As already mentioned with reference to said first exemplary embodiment, said graphical annotation information may be given by body marker symbols, pictograms or polar plots (“clock diagrams”) to be displayed on the monitor screen or display for visually indicating the location of at least one pathological tissue anomaly or lesion or visually indicating the angular orientation of said ultrasound transducer during an ultrasound examination relative to the patient while being slidably moved over the surface of said patient's body.

According to a specific aspect of said third exemplary embodiment, said method may e.g. provide that annotation text and graphical annotation information indicating whether an ultrasound image of the patient's left or right breast, indicating the number of said pathological tissue anomalies or lesions or indicating the radii or angles of their polar coordinates, with the nipple position of said breast being defined as the origin of a polar coordinate system to which said polar coordinates refer, can be selected by rotary knob selection from a number of pull-down menus or scrollable lists.

In more detail, the proposed method according to said third exemplary embodiment may comprise the steps of
displaying annotation texts on the monitor screen or display corresponding to user-selectable parameters including information whether an ultrasound image of the patient's left or right breast is currently being displayed on the monitor screen or display, information indicating the number of said pathological tissue anomalies or lesions, information indicating the radii or angles of their polar coordinates or information indicating the orientation of the ultrasound transducer or said ultrasound transducer's image plane relative to the patient's body during an ultrasound examination of said patient, and
automatically selecting graphical annotation information which corresponds to the selected annotation texts and displaying the selected graphical annotation information on the monitor screen or display, wherein said graphical annotation information comprises a transducer graphic which indicates, relative to the patient's breast, the position and angular orientation of the ultrasound transducer in the aforementioned polar coordinate system with said transducer position and angular orientation being consistent with the displayed annotation text.

According to this method, all these steps, which are to be understood as functions of the graphical user interface as described with reference to said first exemplary embodiment, are interactive in real time such that the annotation text and graphical annotation information immediately respond to a user's command for changing them.

Aside therefrom, a fourth exemplary embodiment of the present invention is dedicated to a workstation or console, programmed with a software which implements a graphical user interface as described with reference to said first exemplary embodiment.

Finally, according to a fifth exemplary embodiment of the present invention, a computer software product configured for performing an image acquisition method as described with reference to said third exemplary embodiment when running on a workstation or console as described with reference to said fourth exemplary embodiment is provided.

### BRIEF DESCRIPTION OF THE DRAWINGS

These and other advantageous aspects of the invention will be elucidated by way of example with respect to the embodiments described hereinafter and with respect to the accompanying drawings therein:

FIG. 1a shows a patient and a physician while executing a breast ultrasound examination;

FIG. 1b illustrates the standard anatomical planes of a patient;

FIG. 2a shows a “clock diagram” for indicating the location of a breast lesion in a woman's left breast and the placement of an ultrasound transducer used for imaging the breast tissue;

FIG. 2b shows an ultrasound image of said breast lesion with text annotation at the bottom that indicates the location of said lesion (pink star) and the orientation of the transducer (gray bar);

FIGS. 3a-e show five pictograms for illustrating different types of breast imaging graphical body markers used by various ultrasound manufacturers;

FIGS. 4a+b show two ultrasound images of female breast tissue for illustrating discordant text annotation and graphical body markers;

FIG. 5 shows a graphical breast annotation user interface of an ultrasound imaging system according to the present invention for interactively modifying an annotation text and graphical annotation information given by a polar plot showing the location and orientation of an ultrasound transducer with respect to the nipple position of a female patient's breast by means of a touch panel's softkeys and/or rotary knob selection, and

FIG. 6 shows a table for linking a set of user-selectable transducer orientation labels which are to be used as displayable annotation texts with a set of discrete transducer orientation angles θ given at increments of 30° for each “full-hour” clock position of a transducer graphic whose current location (indicated by its radius coordinate r in centimeters from the nipple and an angle coordinate φ corresponding to said clock position) is to be graphically displayed in the polar coordinate system of said polar plot.

### DETAILED DESCRIPTION OF THE INVENTION

In the following, the proposed image acquisition device and method according to the present invention will be explained in more detail with respect to special refinements and referring to the accompanying drawings.

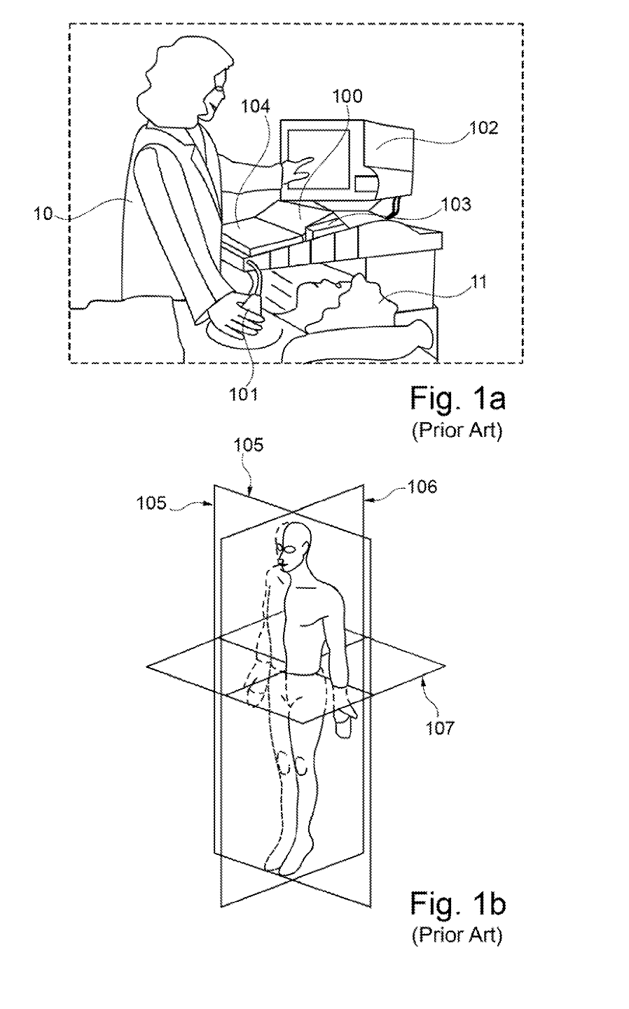

A medical ultrasound system 100 as known from clinical applications typically comprises a control panel 103, a display screen or monitor 102 and an ultrasound tranducer 101. Before an ultrasound-based examination begins, a patient 11 will be instructed to lie on her back (supine), and the attending physician 10 or an assisting radiologist will cover the part of the breast that will be imaged with a gel which lubricates the skin and helps with the transmission of ultrasound waves which are emitted by the transducer (see illustration shown in FIG. 1a).

During the breast ultrasound examination, the sonographer holds the transducer 101 vertically above the breast as shown in FIG. 1 and glides the transducer over the breast while the transducer is emitting sound waves and picking up the back-scattered echoes. A computer will then analyze the echoes and display an image on the monitor 102, and the shape and intensity of the echoes will depend on the density of the breast tissue. If a fluid-filled cyst is being imaged, most of the sound waves will pass through the cyst and emit faint echoes. However, if a solid tumor is being imaged, the sound waves will bounce off the tumor, and the pattern of echoes will be translated by the computer into an image that the radiologist will recognize as indicating a solid mass. Patients may feel a slight pressure from the transducer, but they will not hear the high-frequency sounds.

The image plane of an acquired ultrasound image is usually oriented in such a way that it extends parallel to a patient's axial (transverse) plane 107 or sagittal (lateral) plane 106, although ultrasound images may also be acquired in oblique image planes. Since ultrasound images have a limited field of view, it is crucial to provide annotation information on the image that describes the anatomical location and orientation of the transducer 101. This is especially important in case of sonograms which have been acquired for imaging female breast tissue as there are typically only a few anatomical landmarks to orient the viewer. To allow an easier identification of the respective ultrasound image plane, it is known from the relevant literature to display annotated text or graphical annotation information (such as e.g. transducer orientation labels like “transverse” (TRANS), “axial” (AXIAL), “longitudinal” (LONG), “sagittal” (SAG) or “oblique” (OBL)—to each ultrasound image which is displayed on the ultrasound system's monitor screen 102 or display. An illustration of the standard anatomical planes is shown in FIG. 1b.

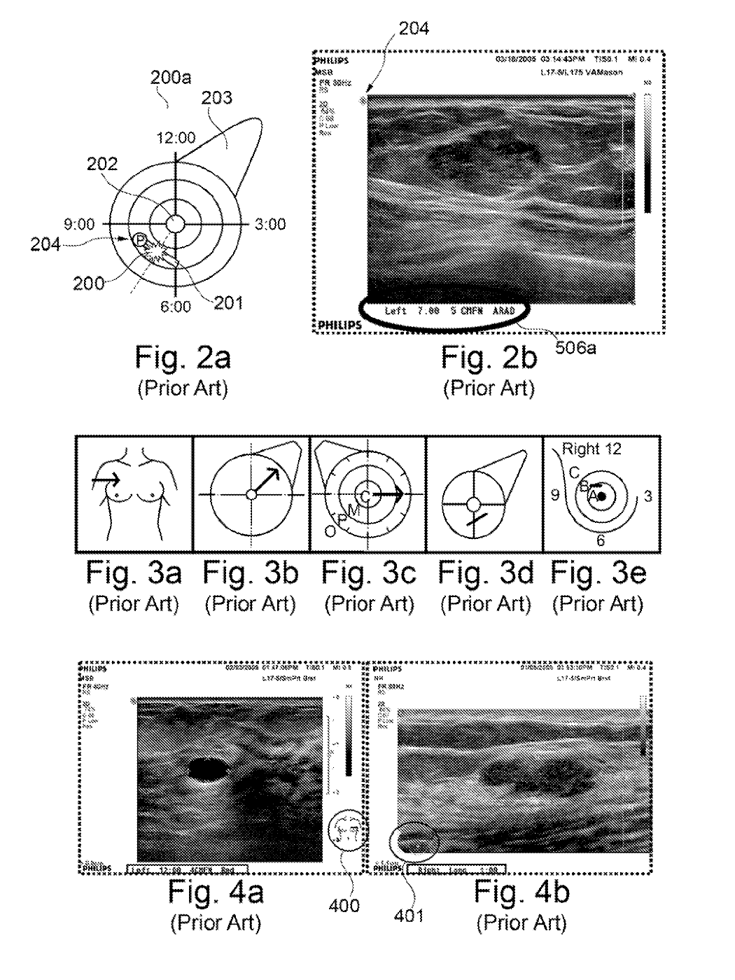

In addition to the transducer orientation, the location of the breast lesion itself must be annotated. A widely accepted method is to use a breast centric coordinate system, in the relevant literature also referred to as a “clock diagram” 200a (such as shown in FIG. 2a). In this polar coordinate system, the center of the clock's face represents a female patient's breast nipple 202. The coordinate position of a lesion can thus be specified as the angular position φ of the “hour hand” (where e.g. “7 o'clock” refers to an angular coordinate or polar angle of φ=210° when the “12 o'clock” position of the “hour hand” is defined as referring to a polar angle of φ=0°) and the radial distance from the nipple position 202 in the centimeters. The tail of breast tissue extending into the armpit, the “axilla” 203 (not to be confused with “axial orientation”), is attached to the clock diagram 200a. The diagram is flipped horizontally to represent the patient's right breast.

An ultrasound transducer symbol 201 is placed on the breast as the location of the lesion (in this case, at a polar angle of φ=210° (“7 o'clock”) and at a radial distance of 5 cm from the nipple position 202), and the transducer orientation (shown in FIG. 2a as a rectangular gray bar) is described with the anatomical abbreviations mentioned previously. In addition to the body centric orientations (axial, sagittal, etc.) the clock diagram 200a provides two more “breast-centric” transducer orientations, namely “radial” (RAD) and “anti-radial” (ARAD). The ultrasound transducer symbol 201 is in a radial orientation when its longitudinal axis is parallel to the “hour hand” of the clock, and it is in anti-radial orientation when it is perpendicular to the hour hand.

In FIG. 2b, an ultrasound image of a breast lesion is shown, wherein text annotation 506a at the bottom indicates the location of said lesion (pink star) and the orientation of an ultrasound transducer (gray bar). Said lesion is in a female patient's left breast, located along a radial line extending from the nipple to the 7 o'clock position at a radial distance of 5 centimeters from the nipple (CMFN). The transducer is placed with an anti-radial orientation, in example perpendicular to the 7 o'clock radial line. The “Circle P” annotation 204 at the end of the transducer graphic in FIG. 2a represents the left side of the image in FIG. 2b, as shown by the “circle P” symbol 204 at the top left corner of the image in FIG. 2b.

Text annotation is traditionally accomplished by use of a keyboard 104 on the ultrasound imaging system 100 to manually type in text and attach it at a selectable position of an image. Alternatively, a touch panel (not shown in FIG. 1a) can be used to choose preselected text phrases. However, editing the text still requires the use of a keyboard.

An alternative annotation method that does not use text annotations is to use a graphical “body marker” (see FIGS. 3a-e) to visually indicate the location of a lesion and transducer orientation. FIGS. 3a-e show five examples of various body markers for breast imaging, most of which are similar to the clock diagram 200a. The position of ultrasound transducer 101 on the breast is thereby indicated with a line or arrow 201 (ultrasound transducer symbol) on the diagram. A user must place the transducer line/arrow on the clock diagram 200a (breast graphic) with the correct position and orientation. This is typically accomplished with the use of a trackball (transducer position) and a rotary knob (transducer orientation). Frequently, both text annotation and graphical body markers are used together on the same image, and therein lies the source of a common problem. Because there are two separate user interfaces—a keyboard or touch screen for text annotation and a trackball for the body marker, this creates extra steps for the user and creates frequent opportunities for errors and inconsistencies between the annotation text and body markers, as shown in the ultrasound breast images depicted in FIGS. 4a+b. Therein, FIG. 4a is falsely labeled “RAD” (radial) although body marker 400 actually shows an “ARAD” (anti-radial) orientation, which means an orientation normal to the plane of the polar coordinate system represented by clock diagram 200a of FIG. 2a. As depicted, FIG. 4b is labeled “LONG” (longitudinal), whereas body marker 401 actually shows a “TRANS” (transversal) orientation. These discrepancies are common since both user interfaces (the one which is used for inputting the annotation text and the one which is used for inputting the body markers) are independent.

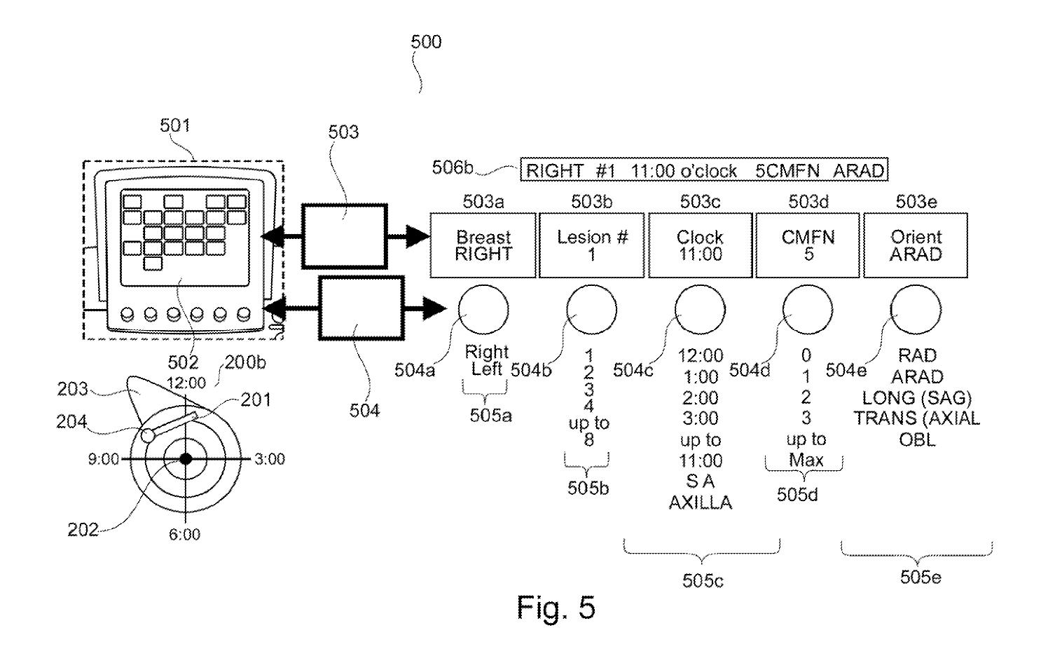

According to a first exemplary embodiment of the present invention, the claimed graphical user interface preferably consists of rotary knobs 504 and a touch screen 502 such as the one used on the touch panel 501 and shown in FIG. 5. The rotary knobs control both the text annotation on the screen and the placement and orientation of the transducer graphic on the body marker 200b shown on the screen. For breast ultrasound imaging, the annotation parameters are “breast” (or side), “lesion number”, “clock position of the lesion”, “CMFN” (i.e., the radial distance of the transducer from the nipple of a female breast to be non-invasively examined, given in centimeters), and “ORIENT” (i.e., the transducer orientation angle). Each knob (in the following more precisely referred to by reference numbers 504a-e) controls the text display (screen text) 506a or 506b of one parameter on the screen 502, and the allowable text value 503a, 503b, 503c, 503d or 503e can be selected from a list 505a, 505b, 505c, 505d or 505e for each parameter.

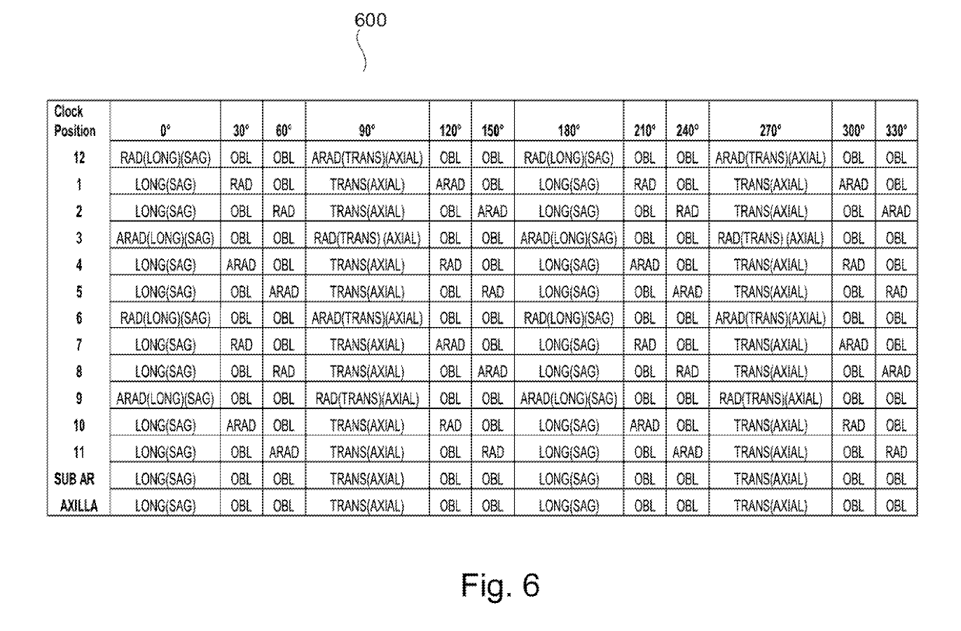

Rotation of the rotary knobs 504 to select the respective text annotation thereby also selects the corresponding correct body marker graphic 200b and controls the position and orientation of the ultrasound transducer symbol 201 (transducer graphic) on the body marker. Rotation of a breast selection knob 504a selects the right or left breast body marker graphic 503a. Rotation of a transducer angle coordinate selection knob 504c (also referred to as “clock knob”) and transducer radius coordinate selection knob 504d (also referred to as “CMFN knob”) move the position of the transducer graphic 201 on the body mark, and a transducer orientation selection knob 504e (also referred to as “ORIENT knob”) rotates said transducer graphic to the correct transducer orientation angle. The user-selectable annotation texts 505a-e (e.g. given by a set of transducer orientation labels 505e such as “RAD (LONG) (SAG)”, “ARAD (LONG) (SAG)”, “LONG (SAG)”, “RAD (TRANS) (AXIAL)”, “ARAD (TRANS) (AXIAL)”, “TRANS (AXIAL)”, “RAD”, “ARAD”, and “OBL”) and the transducer orientations angles for all clock positions 505c are thereby linked by a table 600 as depicted in FIG. 6.

In the present application, unlike known solutions from the prior art, a displayed body marker 200b (such as e.g. a clock diagram) is always consistent to a text annotation 506a or 506b which has been selected or entered by a user. This also applies to the placement of the ultrasound transducer symbol 201 within the clock diagram 200b and the transducer symbol orientation shown in this clock diagram.

### Applications of the Present Invention

The present invention can advantageously be applied in the scope of imaging applications, such as e.g. in ultrasound breast imaging, where it is beneficial to rapidly and consistently input, modify and display annotation text to be linked with, attached to and visualized in a single image or in a set of images displayed on a monitor screen of a graphical user interface. The proposed system and method are especially intended for being applied in a medical workstation, user interface or console, particularly in those dedicated to ultrasound breast imaging.

Although the specific embodiment described in this application disclosure merely refers to 2D breast imaging, the proposed user interface and method can also beneficially be applied to 3D breast imaging and, aside from ultrasound imaging, to any other kind of clinical and non-clinical imaging applications and user interfaces.

While the present invention has been illustrated and described in detail in the drawings and in the foregoing description, such illustration and description are to be considered illustrative or exemplary and not restrictive, which means that the invention is not limited to the disclosed embodiments. Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage. A computer program may be stored/distributed on a suitable medium, such as e.g. an optical storage medium or a solid-state medium supplied together with or as part of other hardware, but may also be distributed in other forms, such as e.g. via the Internet or other wired or wireless telecommunication systems. Furthermore, any reference signs in the claims should not be construed as limiting the scope of the present invention.

Table of used reference numbers and their meaning

| | |
|---|---|
| 10 | Physician |
| 11 | Patient |
| 100 | Ultrasound imaging system (conventional) |
| 101 | Ultrasound transducer |
| 102 | Monitor |
| 103 | Control panel |
| 104 | Keyboard |
| 105 | Coronal plane or Frontal plane |
| 106 | Sagittal plane or Lateral plane |
| 107 | Axial plane or Transverse plane |
| 200 | Suspicious anatomy structure (e.g. a tumor or lesion) |
| 200a | Clock diagram serving as a body marker of the patient's left breast |
| 200b | Clock diagram serving as a body marker of the patient's right breast |
| 201 | Ultrasound transducer symbol |
| 202 | Nipple position |
| 203 | Axilla |
| 204 | Circle “P” annotation or symbol |
| 400 | Body marker showing an ARAD orientation while the image is |
| | labelled RAD |
| 401 | Body marker showing a TRANS orientation while the image is |
| | labelled LONG |
| 500 | Ultrasound imaging system according to the present invention |
| 501 | Touch panel |
| 502 | Touch screen |
| 503 | Touch screen display |
| 503a | Screen text (“RIGHT”), selected by Breast knob 504a |
| 503b | Screen text (“1”), selected by Lesion number knob 504b |
| 503c | Screen text (“11:00”), selected by Clock knob 504c |
| 503d | Screen text (“5”), selected by CMFN knob 504d |
| 503e | Screen text (“ARAD”), selected by Orient knob 504e |
| 504 | Rotary knobs used for selecting a text value or number (by rotation |
| | of Breast knob 504a or Lesion number knob 504b), positioning a |
| | transducer graphic on a body marker (by rotation of Clock knob |
| | 504c and CMFN knob 504d) and orienting said transducer graphic |
| | (by rotation of Orient knob 504e) |
| 504a | Breast knob 504a |
| 504b | Lesion number knob 504b |
| 504c | Clock knob 504c |
| 504d | CMFN knob 504d |
| 504e | Orient knob 504e |
| 505a | List of screen texts (“Right” or “Left”) from which a screen text is |
| | selectable by Breast knob 504a |
| 505b | List of screen texts (“1”, “2”, “3”, “4”, . . . , “8”) from which a |
| | screen text is selectable by Lesion number knob 504b |
| 505c | List of screen texts (“12:00”, “1:00”, “2:00”, “3:00”, . . . , “11:00”, |
| | “SA”, “Axilla”) from which a screen text is selectable by Clock |
| | knob 504c, where “SA” stands for “sub-areolar” |
| 505d | List of screen texts (“0”, “1”, “2”, “3”, . . . , “Max”) from which a |
| | screen text is selectable by CMFN knob 504d, which depends on |
| | the clock position |
| 505e | List of screen texts (“RAD”, “ARAD”, “LONG (SAG)”, “TRANS |
| | (AXIAL)”, “OBL”) from which a screen text is selectable by Orient |
| | knob 504e (Note: list order depends on clock position, cf. table in |
| | FIG. 6) |
| 506a | Displayed screen text (“LEFT 7:00 5 CMFN ARAD”), selected by |
| | rotary knobs 504a and 504c-e as an annotation text |
| 506b | Another displayed screen text (“RIGHT #1 11:00 o'clock 5 CMFN |
| | ARAD”), selected by rotary knobs 504a-e as a further annotation text |
| 600 | Table: Transducer orientation labels versus Transducer orientation |
| | angle for all clock positions (Note: 1. Setups should allow the user |
| | to choose either LONG and/or SAG and AXIAL terminology |
| | according to their preference. 2. A similar table can be constructed |
| | that includes clock positions at the “half hour” (e.g. 2:30) and |
| | transducer angles in 15° increments.) |

1. A graphical user interface (501) for enabling input, modification, display, outfading and cancellation of annotation text and graphical annotation information which, when being selected by a user, are linked with the image data of at least one image on an imaging system's monitor screen or display (102) and displayed at user-definable positions within a region of interest of said at least one image and which, when being deselected by the user, are cancelled or faded out,
wherein the annotation text and said graphical annotation information are linked and depend on each other.

2. The graphical user interface (501) according to claim 1,
wherein the graphical user interface (501) is configured such that, if a specific annotation text is entered, selected or changed by the user, a corresponding graphical annotation information is automatically selected or accordingly modified so as to be consistent with the selected annotation text and displayed together with this selected annotation text on the monitor screen or display (102) and if a specific graphical annotation information is selected or changed by the user, a corresponding annotation text is automatically selected or accordingly modified so as to be consistent with the user-selected graphical annotation information and displayed together with this user-selected graphical annotation information on the monitor screen or display (102).

3. The graphical user interface (501) according to claim 2, configured such that said annotation text and graphical annotation information can be selected or deselected by activating or deactivating softkeys on a touch screen (502) or by using at least one rotary knob (504a-e) for selecting a desired annotation text or graphical annotation information from at least one pull-down menu or scrollable list (505a-e).

4. The graphical user interface (501) according to claim 3, applied in the scope of a breast ultrasound imaging application where said annotation text is given by at least one text label (503e) for indicating the orientation of an ultrasound transducer (101) or said ultrasound transducer's image plane relative to a patient's body during an ultrasound examination of said patient.

5. The graphical user interface (501) according to claim 4, where said graphical annotation information is given by body marker symbols, pictograms or polar plots to be displayed on the monitor screen or display (102) for visually indicating the location of at least one pathological tissue anomaly or lesion or visually indicating the angular orientation of said ultrasound transducer (101) during an ultrasound examination relative to the patient while being slidably moved over the surface of said patient's body.

6. The graphical user interface (501) according to claim 5, configured such that annotation text and graphical annotation information indicating whether an ultrasound image of the patient's left or right breast (503a), indicating the number of said pathological tissue anomalies or lesions (503b) or indicating the radii or angles of their polar coordinates (503c+d), with the nipple position of said breast being defined as the origin of a polar coordinate system to which said polar coordinates refer, can be selected by rotary knob selection from a number of pull-down menus or scrollable lists (505a-e).

7. An ultrasound imaging system for acquiring, processing and displaying image data to be visualized on a monitor screen or display (102), said imaging system comprising a graphical user interface (501) according to claim 1.

8. A method for enabling input, modification, display, outfading and cancellation of annotation text and graphical annotation information which, when being selected by a user, are linked with the image data of at least one image on an imaging system's monitor screen or display (102) and displayed at user-definable positions within a region of interest of said at least one image and which, when being deselected by the user, are cancelled or faded out,
wherein the annotation text and said graphical annotation information are linked and depend on each other.

9. The method according to claim 8, wherein, if a specific annotation text is entered, selected or changed by the user, a corresponding graphical annotation information is automatically selected or accordingly modified so as to be consistent with the selected annotation text and displayed together with this selected annotation text on the monitor screen or display (102) and if a specific graphical annotation information is selected or changed by the user, a corresponding annotation text is automatically selected or accordingly modified so as to be consistent with the user-selected graphical annotation information and displayed together with this user-selected graphical annotation information on the monitor screen or display (102).

10. The method according to claim 9, wherein said annotation text and graphical annotation information can be selected or deselected by activating or deactivating softkeys on a touch screen (502) or by using at least one rotary knob (504a-e) for selecting a desired annotation text or graphical annotation information from at least one pull-down menu or scrollable list (505a-e).

11. The method according to claim 10, applied in the scope of a breast ultrasound imaging application where said annotation text is given by at least one text label (503e) for indicating the orientation of an ultrasound transducer (101) or said ultrasound transducer's image plane relative to a patient's body during an ultrasound examination of said patient.

12. The method according to claim 11, where said graphical annotation information is given by body marker symbols, pictograms or polar plots to be displayed on the monitor screen or display (102) for visually indicating the location of at least one pathological tissue anomaly or lesion or visually indicating the angular orientation of said ultrasound transducer (101) during an ultrasound examination relative to the patient while being slidably moved over the surface of said patient's body.

13. The method according to claim 12, wherein annotation text and graphical annotation information indicating whether an ultrasound image of the patient's left or right breast (503a), indicating the number of said pathological tissue anomalies or lesions (503b) or indicating the radii or angles of their polar coordinates (503c+d), with the nipple position of said breast being defined as the origin of a polar coordinate system to which said polar coordinates refer, can be selected by rotary knob selection from a number of pull-down menus or scrollable lists (505a-e).

14. The method according to claim 13, comprising the steps of
selecting (S1a) and displaying (S1b) annotation texts on the monitor screen or display (102) corresponding to user-selectable parameters including information whether an ultrasound image of the patient's left or right breast (503a) is currently being displayed on the monitor screen or display (102), information indicating the number of said pathological tissue anomalies or lesions (503b), information indicating the radii or angles of their polar coordinates (503c+d) or information indicating the orientation of the ultrasound transducer (101) or said ultrasound transducer's image plane relative to the patient's body during an ultrasound examination of said patient, and
automatically selecting (S2a) graphical annotation information which corresponds to the selected annotation texts and displaying (S2b) the selected graphical annotation information on the monitor screen or display (102), wherein said graphical annotation information comprises a transducer graphic (201) which indicates, relative to the patient's breast, the position and angular orientation of the ultrasound transducer (101) in the aforementioned polar coordinate system with said transducer position and angular orientation being consistent with the displayed annotation text.

15. A workstation or console, programmed with a software which implements a graphical user interface (501) according to claim 1.
